SEQUENCE LISTING (13 sequences)

SEQ ID NO: 1 - protein, 560 aa, Artificial
  recombinant, detoxified form of diphtheria toxin
MSRKLFASILIGALLGIGAPPSAHAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 2 - DNA, 1683 bp, Artificial
  recombinant, detoxified form of diphtheria toxin
gtgagcagaaaactgtttgcgtcaatcttaataggggcgctactggggataggggccccaccttcagcccatgcaggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagagttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcgtctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcgtcggattccataggcgttcttgggtaccagaaaacagtagatcacaccaaggttaattctaagctatcgctattttttgaaatcaaaagctga

SEQ ID NO: 3 - DNA, 27 bp, Artificial
  forward primer
gcaggatcccccgcgtattctccgggg

SEQ ID NO: 4 - DNA, 26 bp, Artificial
  reverse primer
cgctgcagtcagcttttgatttctag

SEQ ID NO: 5 - DNA, 38 bp, Artificial
  forward primer
gcgagatctttaggatagctttacctaattattttatg

SEQ ID NO: 6 - DNA, 40 bp, Artificial
  reverse primer
cgccatgcggccgctcagcttttgatttcaaaaaatagcg

SEQ ID NO: 7 - DNA, 27 bp, Artificial
  forward primer
gcaggatcccccgcgtattctccgggg

SEQ ID NO: 8 - DNA, 30 bp, Artificial
  forward primer
caaccatttcttaaagacgggtatgctgtc

SEQ ID NO: 9 - DNA, 47 bp, Artificial
  forward primer
cgttgcagagctatagacgatgcgaccactttttgtcgccctaaatc

SEQ ID NO: 10 - DNA, 33 bp, Artificial
  forward primer
cggaaaataaggatgcattgcagagctatagac

SEQ ID NO: 11 - DNA, 28 bp, Artificial
  forward primer
gtaactttttgtaaacctaaatctcctg

SEQ ID NO: 12 - DNA, 28 bp, Artificial
  forward primer
gaaatttcgtcgaactccataggcgttc

SEQ ID NO: 13 - DNA, 36 bp, Artificial
  forward primer
cagaaaacagtagataaaaccaaggttaattctaag

(19) United States
(12) Patent Application Publication
(10) Pub. No.: US 2017/0029474 A1
(43) Pub. Date: Feb. 2, 2017

(54) POLYPEPTIDE AND ANTI-TUMOR AGENT

(71) Applicant: OSAKA UNIVERSITY, Osaka (JP)

(72) Inventor: Eisuke MEKADA, Osaka (JP)

(21) Appl. No.: 15/112,756

(22) PCT Filed: Oct. 8, 2014
(86) PCT No.: PCT/JP2014/076945

§ 371 Date: Jul. 20, 2016

(30) Foreign Application Priority Data
Jan. 21, 2014 (JP) 2014-008675

(51) Int. Cl. C07K 14/34; A61K 38/16; A61K 45/06

(52) U.S. Cl. CPC C07K 14/34; A61K 45/06; A61K 38/164; A61K 38/00

(57) ABSTRACT

Provided is a polypeptide comprising an R domain of CRM197 and comprising one or more amino acid substitutions or deletions at positions selected from the group consisting of 391, 460, 466, 467, 468, 472, 507, and 520 of the R domain.

### CROSS REFERENCE TO RELATED APPLICATIONS

This application claims the benefit of priority to Japanese Patent Application No. 2014-008675, filed on Jan. 21, 2014, the entire contents of which are incorporated by reference herein.

### TECHNICAL FIELD

Technical Field

The present invention relates to a polypeptide and an antitumor agent.

Background Art

HB-EGF is a heparin-binding growth factor. Growth factors of the EGF family include not only HB-EGF, but also EGF, transforming growth factor (TGF-α), amphiregulin (AR), epiregulin, and the like. These molecules bind to the EGF receptor and its family (ErbB family) molecules to promote the phosphorylation of the receptor. More specifically, HB-EGF binds to EGF receptors ErbB1 and ErbB4. HB-EGF is expressed in various tissues, and high expression of the mRNA is observed in the lung, skeletal muscle, and heart. Further, it has been reported that HB-EGF is highly expressed in various cancer tissues and sites of inflammation. There are reports that HB-EGF greatly promotes growth of fibroblasts, smooth muscle cells, or keratinocytes. It has also been reported that HB-EGF plays an important role in the morphogenesis and regeneration of organisms as well as being intimately involved in the growth, invasion, metastasis, and anti-cancer drug resistance of cancer cells. HB-EGF is synthesized as a membrane-anchored molecule (proHB-EGF) on the cell membrane and cleaved by protease on the cell surface to yield soluble HB-EGF (sHB-EGF). This sHB-EGF mainly serves as a growth factor, but there is also a report that proHB-EGF acts as a growth factor in a membrane-anchored form (Non-patent Literature 1 to 3).

Diphtheria toxin (DT), which is produced by Corynebacterium diphtheriae, is a 535-amino-acid protein with a molecular weight of about 58.4 kDa. DT is composed of fragment A and fragment B. Conformationally, DT includes a catalytic domain (C domain), a transmembrane domain (T domain), and a receptor binding domain (R domain). Fragment A corresponds to the C domain, and fragment B is composed of T and R domains (FIG. 1). After binding to proHB-EGF in the R domain, DT enters the cell and reaches acidic vesicles. Then, the T domain is inserted into the acidic vesicle membrane to facilitate passage of the C domain through the vesicle membrane. Finally, the C domain reaches the cytoplasm and ADP-ribosylates peptide elongation factor (EF2) to inactivate EF2, resulting in the inhibition of protein synthesis in the cell (Non-patent Literature 4).

CRM197 (Cross reaction material) has G52E mutation in DT. This mutation inactivates the ADP-ribosylation activity, thus causing toxicity as DT to be lost; however, since CRM197 has the same R domain as that of DT, it binds to proHB-EGF in the same manner as DT. The R domain of DT and the R domain of CRM197 bind to the EGF-like domain of proHB-EGF. Therefore, DT and CRM197 bind to sHB-EGF, as well. Once DT or CRM197 binds to sHB-EGF, the sHB-EGF cannot bind to the EGF receptor, resulting in suppression of the cell growth effect of sHB-EGF. CRM197 can thus be used as an HB-EGF inhibitor (Non-patent Literature 5 and Patent Literature 1 and 2).

Studies are in progress to develop CRM197 as an anti-cancer agent by utilizing the HB-EGF inhibitory effect of CRM197. In nonclinical tests to date, CRM197 is reported to strongly suppress tumorigenesis of human cancer cells inoculated into nude mice, and, furthermore, clinical trials for an anti-cancer agent containing CRM197 as an active ingredient have been performed (Non-patent Literature 6 and 7).

### CITATION LIST

### Patent Literature

PTL 1: JP2006-232761A

PTL 2: JP2004-155776A

### Non-Patent Literature

NPL 1: Raab, G. and M. Klagsbrun, Heparin-binding EGF-like growth factor. Biochim Biophys Acta, 1997. 1333 (3): p. F179-99.

NPL 2: Iwamoto, R., at al., Heparin-binding EGF-like growth factor and ErbB signaling is essential for heart function. Proc Nati Acad Sci USA, 2003. 100 (6): p. 3221-6.

NPL 3: Higashiyama, S., at al., The membrane protein CD9/DRAP 27 potentiates the juxtacrine growth factor activity of the membrane-anchored heparin-binding EGF-like growth factor. J Cell Biol, 1995. 128 (5): p. 929-38.

NPL 4: Collier, R. J., Understanding the mode of action of diphtheria toxin: a perspective on progress during the 20th century. Toxicon, 2001.39 (11): p. 1793-803.

NPL 5: Mitamura, T., at al., Diphtheria toxin binds to the epidermal growth factor (EGF)-like domain of human hepatin-binding. EGF-like growth factor/diphtheria toxin receptor and inhibits specifically its mitogenic activity. J Biol Chem, 1995. 270 (3): p. 1015-9.

NPL 6: Miyamoto, S., et. al., Heparin-binding EGF-like growth factor is a promising target for ovarian cancer therapy. Cancer Res, 2004. 64 (16): p. 5720-7.

NPL 7: Miyamoto, S., at al., Heparin-binding epidermal growth factor-like growth factor as a novel targeting molecule for cancer therapy. Cancer Sci, 2006. 97 (5): p. 341-7

### SUMMARY OF INVENTION

### Technical Problem

An object of the present invention is to provide a polypeptide and an antitumor agent that more strongly inhibit the binding of HB-EGF to the EGF receptor.

### Solution to Problem

The present inventor conducted extensive research on the preparation of a molecule that suppresses the growth factor effect of HB-EGF more strongly than CRM197, and found that the binding between HB-EGF and the EGF receptor can be more strongly inhibited by an amino acid substitution in at least specific position of the R domain of CRM197.

The present invention provides the following polypeptide and antitumor agent.

Item. 1. A polypeptide comprising an R domain of CRM197 and comprising one or more amino acid substitutions at positions selected from the group consisting of 391, 460, 466, 467, 468, 472, 507, and 520 of the R domain.

Item 2. The polypeptide according to Item 1, which comprises one or more amino acid substitutions at positions selected from the group consisting of 391, 460, 466, 467, 468, and 472 of the R domain.

Item 3. The polypeptide according to Item 1 or 2, which comprises at least one member selected from the group consisting of H391K, R460H, G466D, D467A, V468T, R472K, D507N, and H520K in. the R domain of CRM197.

Item. 4. The polypeptide according to any one of Items 1 to 3, wherein the polypeptide is a modified CRM197 comprising a C domain, a T domain, and the R domain of CRM197.

Item 5. An antitumor agent comprising the polypeptide according to any one of Items 1 to 4.

Item 6. The antitumor agent according to Item 5, wherein an antitumor agent is bound to the polypeptide according to any one of Items 1 to 4.

Item 7. A pharmaceutical composition comprising an effective amount of the polypeptide according to any one of Items 1 to 4 and a pharmaceutical carrier.

Item 8. A method for treating a tumor, comprising administering an effective amount of the polypeptide according to any one of Items 1 to 4 to a subject.

Item 9. Use of the polypeptide according to any one of Items 1 to 4 for the production of an antitumor agent.

### BRIEF DESCRIPTION OF DRAWINGS

FIG. 1 shows the primary structure of CRM197. CRM197 is composed of fragment A and fragment B. The fragment B is structurally divided into the T (transmembrane) domain and R (receptor-binding) domain.

FIG. 2 shows the suppression of the HB-EGF-dependent growth of DER cells by CRM197 or a recombinant R domain protein. DER cells were cultured in the presence of 10 ng/mL of HB-EGF and various concentrations of CRM197 or a recombinant R domain protein for 40 hours, and the number of cells was counted. FIG. 2(A) shows a comparison of CRM197 with MBP-R (WT). FIG. 2(B) shows a comparison of MBP-R (WT) with R (WT).

FIG. 3 shows the suppression of the HB-EGF-dependent growth of DER cells by MBP-R mutants and a comparison of the mutants having an increased inhibitory effect. In FIG. 3, MBP-R (466-468) represents MBP-R (G466D/D467A/V468T).

FIG. 4 shows the suppression of the HB-EGF-dependent growth of DER cells by MBP-R mutants ((A) V4681 mutant, (B) D507N mutant, and (C) H520K mutant).

FIG. 5 shows effects of CRM197 or MBP-R mutants on the TGF-α-dependent growth of DER cells. DER cells were cultured in the presence of 3 ng/mL of TGF-α and various concentrations of CRM197, MBP-R (WU), or an MBP-R mutant. In FIG. 5, MBP-R (466-468) represents MBP-R (G466D/D467A/V468T).

FIG. 6 shows the inhibitory effects of R-domain mutants on the growth of BRL cells (A to C) and BRLH cells (D and E) in a three-dimensional (3D) culture. FIG. 6 (A) to FIG. 6 (C) show the effects of MBP-R mutants on BPI cells stimulated by recombinant. HB-EGF. CRM197, MBP-R (WT), or an MBP-R mutant (5 μM each), and recombinant HB-EGF (30 ng/ML) were added to a culture medium and cultured in 3D conditions. FIG. 6 (D) and FIG. 6 (E) show the effects of MBP-R mutants on BRLH cell growth. BRLH cells were cultured in the presence of CRM197, MBP-R PM, or an MBP-R mutant (5 μM each). In FIG. 6, 466-468 represents G466D/D467A/V468T. N.S means not significant. *P<0.05, **P<0.005, ***P<0.0005.

FIG. 7 shows the suppression of the HB-EGF-dependent growth of DER cells by a CRM197 mutant. DER cells were cultured in the presence of 10 ng/ml, of HB-EGF and various concentrations of CRM197 or CRM197 (R460H) for 40 hours, and the number of cells was counted.

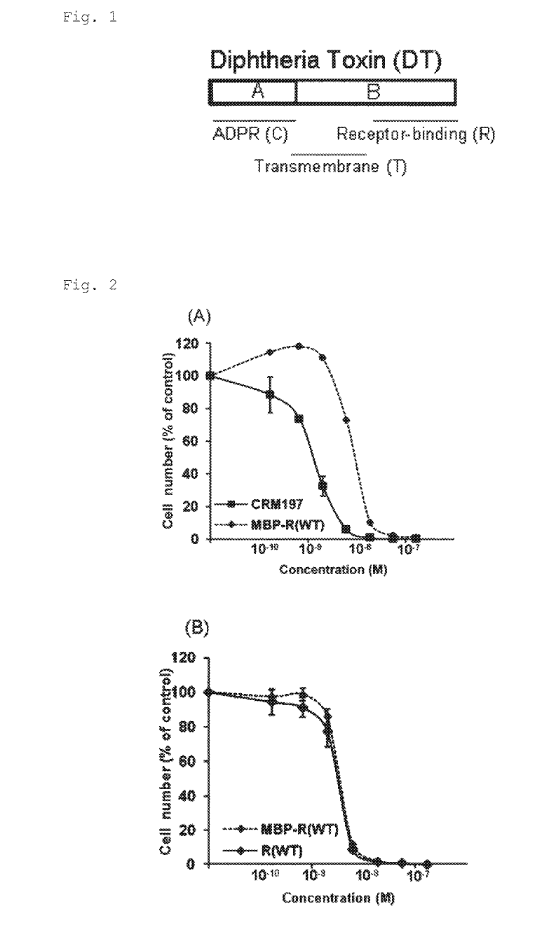

### DESCRIPTION OF EMBODIMENTS

As used herein, the singular forms “a,” “an,” and. “the” are intended to include the singular and the plural unless otherwise indicated herein or clearly contradicted by context.

As used herein, the amino acids of CRM197 are numbered starting with the amino acid ((Gly)) at position 26 in the amino acid sequence of SEQ ID NO: 1 as number 1, excluding the signal sequence (1 to 25).

In SEQ ID NO: 1, the three domains of CRM197 consist of the following amino acids:
C domain: 1-193
T domain: 194-379
R domain: 380-535.

The polypeptide of the present invention comprises one or more amino acid substitutions at positions selected from the group consisting of 391, 460, 466, 467, 468, 472, 507, and 520 of the R domain of CRM197. The preferred positions of substitutions are positions 391, 460, 466, 467, 468, and 472. The most preferred substitutions are H391K, R460H, G466D/D467A/V468T, R472K, D507N, and H520K.

An amino acid substitution at a specific position in the polypeptide comprising the R domain of CRM197 can be introduced by performing site-directed mutagenesis using a. Site-Directed Mutagenesis System Mutan (registered trademark)-Super Express Km kit (TAKARA RIO Inc.) or the like on DNA encoding the polypeptide, or by replacing a specific base by another base with respect to the gene by recombinant PCR (polymerase chain reaction) (PCR protocols, Academic Press, New York, 1990), SOE (splicing by overlap extension)-PCR (Gene, 77, 61, 1989), or the like.

The amino acid sequence of CRM197 is shown in SEQ ID NO: 1, and the DNA sequence encoding CRM197 is shown in SEQ ID NO: 2. Introduction of a mutation, such as a substitution, in the polypeptide can be performed using the DNA. sequence of SEQ ID NO: 2

The site into which such an amino acid substitution mutation is introduced is a site that allows for stronger activity to inhibit the binding between HB-EGF and the EGF receptor than the activity of the wild-type (WT) R domain, into which no mutation is introduced. However, the polypeptide of the present invention also includes mutants further comprising an amino acid substitution or deletion at a position with little or no impact on such inhibitory activity other than the positions above as long as they have inhibitory activity more excellent than the wild-type R domain.

The polypeptide of the present invention comprises an R domain (a modified R domain) comprising an amino acid substitution at a specific position as described above. The polypeptide of the present invention may consist of the R domain or may further comprise the T and C domains of CRM197 or the like linked thereto. Therefore, the polypeptide of the present invention may be a modified CRM197 comprising a modified R domain. As shown in the Examples, MBP-R, in which a maltose binding protein (MB?) is linked to a modified. R domain, has inhibitory activity similar to that of the R domain. Since the inhibitory activity is not changed even when another protein is bound to the R domain, the polypeptide of the present invention encompasses a wide range of polypeptides in which another amino acid, peptide, or polypeptide is bound to the N-terminus or C-terminus of the modified R domain. Such a polypeptide can be biotechnologically produced using DNA encoding the polypeptide by a method well known in the art.

Moreover, a known antitumor agent may be chemically bound to the polypeptide of the present invention. Examples of known antitumor agents to be bound to the polypeptide of the present invention include taxol, taxotere, docetaxel, irinotecan, topotecan, doxorubicin, etoposide, CPT-11, camptostar, epothilone, tamoxifen, methoxtrexate, 5FU, temozolomide, cyclophosphamide, SCH66336, R115777, L778, 123, BMS214662, Iressa, Tarceva, antibodies to EGF receptor (EGFR), Gleevec, intron, ara-C, Adriamycin, Cytoxan, gemcitabine, uracil mustard, chlormethine, ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatin, vinblastine, vincristine, vindesine, bleomycin, dactinomycin, daunorubicin, epirubicin, idarubicin, mithramycin, deoxycoformycin, mitomycin-C, and the like.

The antitumor agent of the present invention is effective for treatment of cancer, such as ovarian cancer, breast cancer, prostate cancer, stomach cancer, lung cancer, colorectal cancer, and pancreatic cancer.

The anti-cancer agent of the present invention is effective for treatment of cancer in which the expression of HB-EGF is especially enhanced among growth factors of the EGF family.

The antitumor agent or anti-cancer agent of the present. invention can be formulated from the above active ingredient, or can be formulated by coming the ingredient with a pharmaceutically acceptable pharmaceutical carrier. The pharmaceutical carrier refers to a known pharmaceutically acceptable solid or liquid carrier that is pharmacologically inert and that is useful for formulation for administration of a pharmaceutically active ingredient.

The above therapeutic agent can be administered orally or parenterally (e.g., intravenous, intramuscular, intraperitoneal, subcutaneous or intradermal injection, intrarectal administration, transmucosal administration, or administration via the respiratory tract).

Examples of the dosage form of the pharmaceutical composition suitable for oral administration include, but are not limited to, tablets, granules, capsules, powders, solutions, suspensions, syrups, and the like. Examples of the dosage form of the pharmaceutical composition suitable for parenteral administration include, but are not limited to, injections, drops, suppositories, percutaneous absorbents, and the like.

Types of pharmaceutical additives used for production of the therapeutic agent are not particularly limited and can be suitably selected by those skilled in the art. Examples of additives that can be used include excipients, disintegrators or disintegration aids, binders, lubricants, coating agents, bases, solubilizers or solubilizing agents, dispersants, suspending agents, emulsifiers, buffers, antioxidants, preservatives, tonicity adjusting agents, pH adjusters, solubilizers, stabilizers, and the like. Specific individual ingredients used for these purposes are well known to those skilled in the art.

Examples of pharmaceutical additives that can be used for production of the preparation for oral administration include excipients, such as glucose, lactose, D-mannitol, starch, and crystalline cellulose; disintegrators or disintegration aids, such as carboxymethylcellulose, starch, and carboxymethylcellulose calcium; binders, such as hydroxypropylcellulose, hydroxypropyl methylcellulose, polyvinylpyrrolidone, and gelatin; lubricants, such as magnesium stearate and talc; coating agents, such as hydroxypropyl methylcellulose, sucrose, polyethylene glycol and titanium oxide; and bases, such as Vaseline, liquid paraffin, polyethylene glycol, gelatin, kaolin, glycerin, purified water, and hard fat.

Examples of pharmaceutical additives that can be used for production of the preparation for injection or infusion. include solubilizers or solubilizing agents that can constitute aqueous injections or injections that are dissolved at the time of use, such as distilled water for injection, physiological saline, and propylene glycol; tonicity adjusting agents, such as glucose, sodium chloride, D-mannitol, and glycerin; pH adjusters, such as inorganic acids, organic acids, inorganic bases, and organic bases; and the like.

Although the amount of the active ingredient contained in the therapeutc agent of the present invention varies depending on the dosage form or administration route of the therapeutic agent and cannot unconditionally be defined, it can be generally selected and determined as appropriate from the range of about 0.0001% to 70% in the final preparation.

The therapeutic agent of the present invention can be administered to mammals, including human beings, in particular human beings, as a subject.

Although the dose of the therapeutic agent of the present invention should be appropriately increased or reduced. depending on conditions, such as the age, sex, body weight, and symptoms of a patient and the route of administration, it is such that the amount of the active ingredient per day per adult is preferably about 1 μg to 100 mg, more preferably about 10 μg to 10 mg, and even more preferably about 100 μg to 5 mg. The pharmaceutical in the above dose may be administered in one to several portions a day. The pharmaceutical may be administered once a week over a period of 6 to 8 weeks, may be administered every other day over a period of 2 to 3 weeks, or may be administered daily for 10 to 14 days.

The present invention includes a method for treating a tumor, comprising administering an effective amount of the polypeptide of the present invention to a subject. The present invention also includes use of the polypeptide of the present invention for the production of an antitumor agent.

### EXAMPLES

Examples are given below to illustrate the present invention in more detail; however, the present invention is not limited to these Examples.

### Example 1

### Materials and Methods

Reagents

Recombinant HB-EGF and TGF-α were obtained from R&D Systems (MN, U.S.A.). C-terminal His-tagged HB-EGF lacking the heparin-binding domain was purified as described in the document (Takazaki, R., et al., J Biol Chem, 2004. 279 (45): p. 47335-43).

### Cells and Culture

DER cells were established and maintained by introducing exogenous human EGFR cDNA into bone marrow-derived lymphoblastic 32D cells as previously reported. (Iwamoto, R., K. Handa, and E. Mekada, J Biol Chem, 1999. 274 (36): p. 25906-12.). Buffalo rat liver (BRL) cells and BRL cells (BRLH) that express exogenous human HB-EGF were established and maintained as previously reported (Mizushima, H., et al., J Cell Sci, 2009. 122 (Pt 23): p. 4277-86).

### Glutathione-S-Transferase (GST)-Fusion R Domain

The cDNA of the entire R domain (residues 380-535) amplified by PCR was inserted into the BamHI-EcoRI site of a pGEX-3 vector (GE Healthcare) to transform E. coli JM109. A single colony was inoculated into 40 mL of an LB medium containing 50 μg/mL ampicillin (LB-amp) and grown at 37° C. overnight. The pre-culture medium was inoculated into 500 mL of fresh LB-amp and grown at 37° C. until it reached the bacterial logarithmic growth phase. 0.4 mM isopropyl β-D-1-thiogalactopyranoside (IPTG) was added thereto and cultured at 37° C. for 2 hours. The culture medium was centrifuged to collect E. coli cells. The E. coli cells were washed with solution A (20 mM Tris-HCl, pH of 8.0, 30 mM NaCl, 10 mM EDTA) containing 0.2 mM phenylmethylsulfonyl fluoride (PMSF), collected again by centrifugation, and lysed with solution. A containing lysozyme (40 μg/mL) on ice for 1 hour. Triton-X100 (0.53%) and NaCl (100 mM) were added to the bacterial lysate, followed by incubation on ice for 15 minutes and sonication in the presence of 0.2 mM PMSF for 20 seconds (twice on ice). Centrifugation was performed, and the supernatant was incubated with a glutathione sepharose 4B gel (GE Healthcare) at 4° C. overnight. The gel was washed with solution A, and bound protein was eluted with an elution buffer (100 mM Tris-HCl, pH of 8.0, 6 mM glutathione).

### Thioredoxin (TRX)-Fusion R Domain

The cDNA of the R domain was cloned into the XhoI-EcoRI site of a pThioHis vector (Invitrogen, CA, U.S.A.) to transform E. coli JM109. The ThioHis-fusion protein was purified using an His-Patch ThioFusion™ expression system. (Invitrogen) according to the manufacturer's instructions.

Maltose Binding Protein (MBP)-Fusion R domain

The cDNA of R domain was cloned into the BamHI-PstI site of a pMAL-p2 vector (New England BioLabs Inc., MA, U.S.A) to transform E. coli JM109. The primers used for PCR were as follows (the restriction enzyme sites for cloning are underlined):

(SEQ ID NO: 3)
Fw, 5′ -GCAGGATCCCCCGCGTATTCTCCGGGG-3′.
(SEQ ID NO: 4)
Rv, 5′ -CGCTGCAGTCAGCTTTTGATTTCTAG-3′.

Further, to separate the R domain from MBP, an oligonucleotide encoding the PreSicission Protease (GE Healthcare) recognition sequence (LEVLFQ/GP) was inserted into the BamHI site of the pMAL-p2 vector.

A single colony was inoculated into 40 mL of LB-amp and grown at 37° C. overnight. The pre-culture was inoculated into 500 mL of fresh LB-amp and cultured at 37° C. until it reached the logarithmic growth phase. 1 mM IPTG was further added, followed by culturing at 37° C. for 3 hours. The bacteria were collected by centrifugation and washed with buffer B (20 mM HEPES, pH of 7.3, 150 mM NaCl). The bacteria were collected again by centrifugation, resuspended in buffer B, and disrupted by sonication. The bacterial lysate was clarified by centrifugation, and the supernatant was mixed with an amylose resin (New England BioLabs) at 4° C. overnight. The resin was washed with buffer B, and bound protein was eluted with buffer B containing 100 mM maltose. Release of the R domain from the fusion protein was carried out by incubation with PreSicission Protease. The released MBP and PreSicission Protease were removed by incubation with the amylose resin and glutathione sepharose 4B.

### MBP-R Domain Mutants

MBP-R domain mutants were constructed by site-directed mutagenesis using PCR. The oligonucleotides used are listed in Table 1. Plasmids encoding the MBP-R domain mutants were individually introduced. into E. coli JM109 for transformation. The MBP-R. domain mutants were purified by the aforementioned. method.

### Growth Inhibition Assay

DER cells (1×10⁴ cells) were cultured in a RPMI1640/10% FCS medium containing 10% fetal bovine serum (FES), recombinant HB-EGF (10 ng/ml), heparin (10 μg/ml), penicillin G (100 U/ml), and streptomycin (100 μg/ml) in the presence or absence of various concentrations of MBP-R domain protein for 40 hours. The concentrations of the MBP-R domain protein were 0.22, 0.67, 2, 6.1, 18.5, 55.5, and 167 μM. The cells were cultured using a 96-well plate (100 μL medium/well). The number of DER cells was measured using Cell Count Reagent SF (Nacalai Tesque, Japan) according to the manufacturer's manual. All experiments were performed three times, and data are represented as mean ±s.d.

### Collagen Gel Culture

Ice-cold bovine Type I collagen (Nitta Gelatin; 3 mg/ml), a reconstitution buffer containing 2.2% (w/v) NaHCO₃, 0.2 M HEPES, and 50 mM NaOH; a 10X DMEM-F12 (1:1) medium; and serum-free DMEM in which cells were suspended (5×10⁶ cells/mL) were mixed at an 8:1:1:0.1 ratio, and the mixture was poured into a 24-well dish (0.5 mL/well) as reported previously (Mizushima, H., et al., J Cell Sci, 2009. 122 (Pt 23): p. 4277-86). After incubation at 37° C. for 30 minutes, the collagen gel was covered with 1 mL of DMEM-FBS containing any of CRM197, MBP-R (WT), and MBP-R (mutant), followed by culturing for 1 week. To count the number of cells, incubation with 0.5 ml of 0.5% (w/v) bacterial collagenase (Invitrogen, CA, U.S.A.) was performed at 37° C. in HBS (+) (10 nM HEPES, pH of 7.2, 140 nM NaCl, 4 nM KCl, 1.8 mM CaCl₂, and 1 mM MgCl₂) until the collagen gel was dissolved, and cells were collected by centrifugation. The cells were fixed and stained with a crystal violet solution (0.1% crystal violet, 0.1 M citric acid), and the number of nuclei was counted under a microscope after vortexing. All experiments were performed three times, and data are represented as mean ±s.d.

### Surface Plasmon Resonance

Biacore T200 and Sensor Chip NTA (GE Healthcare) were used for this analysis. The coupling constant of HB-EGF and an R domain mutant was measured according to the manufacturer's instructions. Ni²⁺ was captured at a flow rate of 10 μL/mL on the sensor chip for 60 seconds, and subsequently C-terminal His-tagged recombinant HB-EGF (Δ••-EGF-His) lacking the heparin-binding domain was captured for 180 seconds. Various concentrations of CRM197, MBP-R (WT), or MBP-R (mutant) were allowed to act on the sensor chip at a flow rate of 30 μL/mL for 600 seconds for a binding reaction. Thereafter, dissociation was monitored at a flow rate of 30 μL/mL for 600 seconds. KD and KA values were calculated using Biacore Software (GE Healthcare).

Establishment of C. diphtheriae that Expresses CRM197 Mutant by Means of Triparental Mating

A CRM197 gene and its promoter were obtained from genomic DNA of C. diphtheriae C7hm723 (β197) strain (Eanei, C., T. Uchida, and M. Yoneda, infect immun, 1977. 18 (1): p. 203-9.) by PCR. The primers used are shown below (the restriction enzyme sites for cloning are underlined):

(SEQ ID NO: 5)
Fw, 5′-GCGAGATCTTTAGGATAGCTTTACCTAATTATTTTATG-3′,
(SEQ ID NO: 6)
Rv, 5′-CGCCATGCGGCCGCTCACTTTTGATTTCAAAAAATAGCG-3′.

The amplified DNA was inserted into the BglII-NotI site of a pK-PIM vector (Oram, M., Woolston, J. E., Jacobson, A. D. Holmes, R. K. & Oram, D. M. Bacteriophage-based vectors for site-specific insertion of DNA in the chromosome of Corynebacteria Gene 391, 53-62, (2007).). A CRM197 (R460H) mutant was constructed by site-directed mutagenesis using pK-PIM/CRM197. DH10B/pK-PIM, HB101/prk2013, and C. diphtheriae were individually pre-cultured at 37° C. overnight and mixed at a ratio of DH10B:HB101:C. diphtheriaeC7 (−)=1:1:8. The bacterial mixture pellet, was added to a Brain. Heart Infusion (BHI) agar plate and incubated at 30° C. The bacterial mixture was diluted with a BHI medium and incubated in a complex BHI agar plate containing nalidixic acid (20 μg/mL) and kanamycin (5 μg/mL) at 30° C. for 16 hours. Further, an isolated colony was cultured in a BHI medium containing 20 μg/mL of nalidixic acid and 5 μg/mL of kanamycin to obtain Corynebacterium diphtheriae into which pK-PIM was incorporated.

Preparation and Purification of CRM197 and CRM197 mutant.

CRM197 and a CRM197 mutant were prepared and purified as previously reported (Uchida, T., A. M. Pappenheimer, Jr., and R. Greany, J Biol Chem, 1973. 248 (11): p. 3838-3844).

### Statistical Analysis

Unless otherwise indicated, data were represented as mean ±standard deviation. A statistically significant difference was evaluated using Student's t-test. A value of p<0.05 was considered significant.

### Results

Preparation of Recombinant R Domains of CRM197 and HB-EGF-inhibitory activity

R domains of CRM197 fused to three kinds of expression-purification tags (GST, TRX, and MBP) were constructed and expressed in Escherichia coli. These fusion proteins, called GST-R (WT), TRX-R (WT), and MBP-R (WT), were prepared and purified according to a standard procedure or the manufacturer's instructions. Since the MBP-fusion protein had the highest purity and stablity among the constructed fusion proteins, the MBP-R domain was used for further investigations.

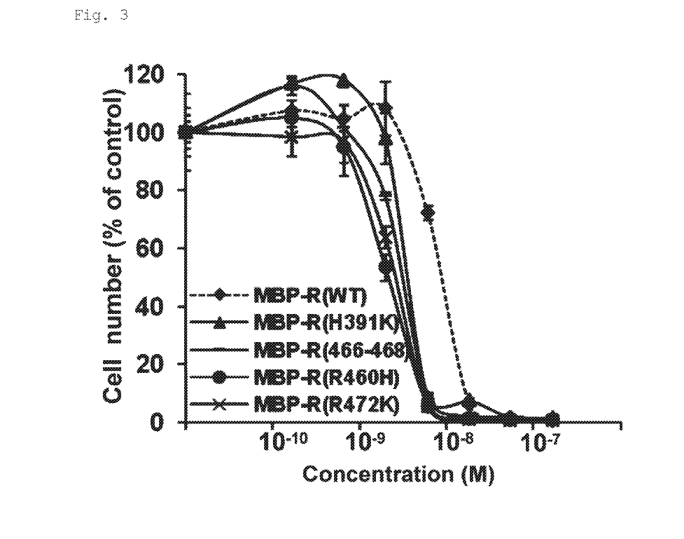

First, it was investigated whether MBP-R (WT) suppresses HB-EGF-dependent cell growth using a DER cell growth assay system. DER cells are derived from IL-3-dependent 32D cells, stably express human EGF receptor (EGFR), and can grow in EGFR ligand-containing media. DER cells show EGFR-ligand-dependent cell growth in the absence of IL-3. Therefore, DER cell growth assay in the presence of HB-EGF allows detection of HB-EGF-inhibitory activity in a test sample in an HB-EGF-specific manner. MBP-R (WT) suppressed the growth of DER cells in a dose-dependent manner (FIG. 2A). However, its inhibitory activity was lower than that of CRM197. The ID₅₀ values, which are defined as the concentration that causes a 50% reduction of the number of cells, in CRM197 and MBP-R (WT) were 1.2 and 7.9 nM, respectively. It was considered that a possible reason why MBP-R (WT) showed inhibitory activity lower than that of CRM197 was that the MBP tag suppressed interaction between HB-EGF and the R domain by steric hindrance; however, this was found to be incorrect. The MBP tag was removed from MBP-R (WT) by using PreSicission Protease (GE Healthcare) to purify R (WT). R and MBP-R (WT) showed similar inhibitory activity (FIG. 2B), indicating that the MBP tag does not interfere with the inhibitory activity of the R domain.

### Inhibitory Activity of R Domain Mutants and Characterization of R Domain Mutants Having Increased Inhibitory Activity

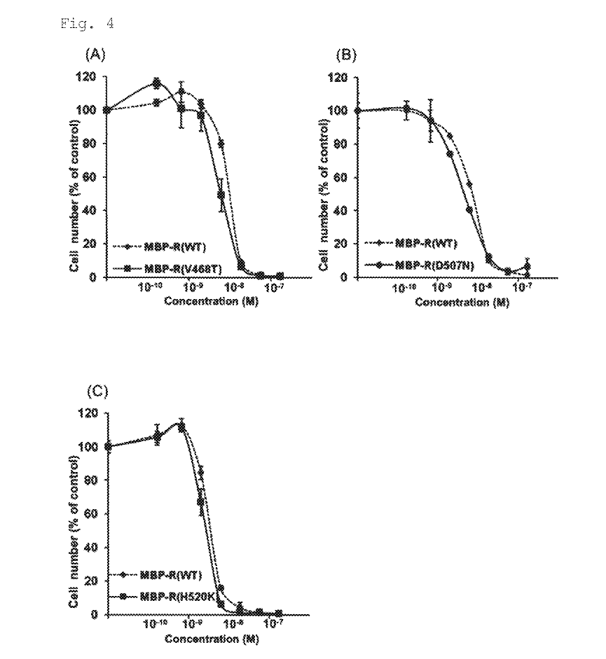

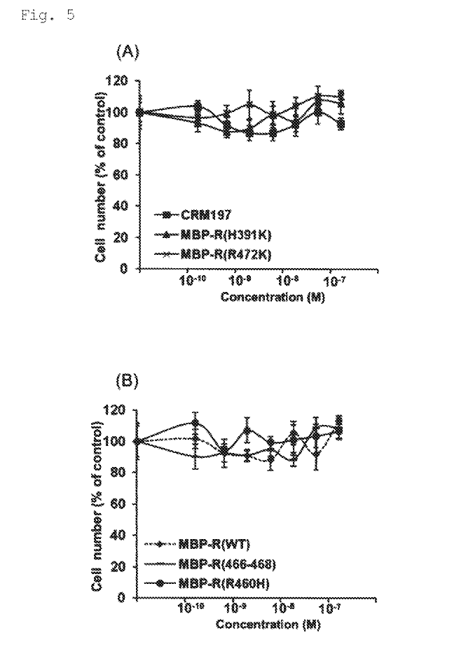

To evaluate the inhibitory activity of R domain mutants against HB-EGF cell growth activity, DER cells were cultured with a certain amount of HB-EGF in the presence of various concentrations of the R domain mutant proteins. Representative data are shown in FIGS. 3 and 4. Table 2 shows the ID₅₀ values of the MBP-R mutants.

Six R domain mutant proteins showed inhibitory activity higher than that of MBP-R (WT). The ID₅₀ value of each mutant compared with CRM197 was as follows: CRM197, 1.2 nM; MBP-R (R460H), 2.5 nM; MBP-R (H391K), 3.1 nM; MBP-R (R472K), 3.2 nM; MBP-R (D507N), 4.5 nM; MBP-R (H520K), 6.0 nM; MBP-R (V468T), 6.2 nM; MBP-R (WT), 7.9 nM.

Next, a mutant having multiple mutations (MBP-R (G466D/D467A/V468T)) was prepared and its inhibitory activity was also investigated. FIGS. 3 and 4 and Table 3 show the results. The ID₅₀ value of the MBP-R (G466D/D467A/V4681) mutant was 2.6 nM, indicating a notable increase in inhibitory activity owing to the multiple mutations.

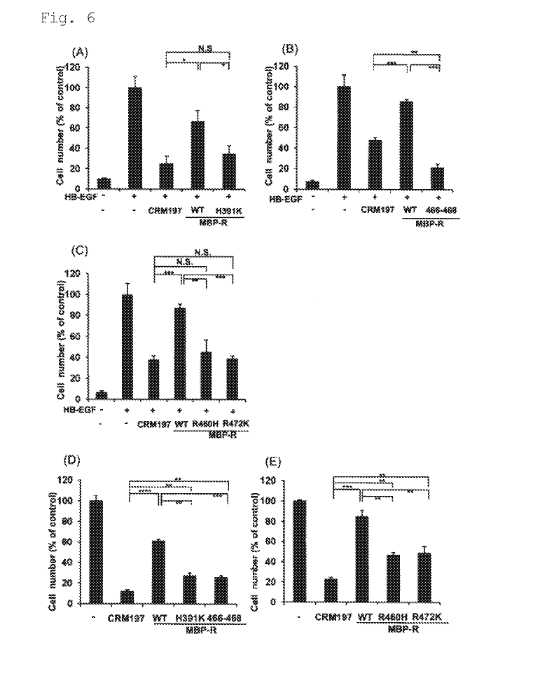

To investigate whether the inhibitory activity of these R domain mutants is specific to HB-EGF, DER cells were cultured in the presence of TGF-α and the R domain mutants. None of these mutants affected the growth of DER cells (FIG. 5), indicating that these R domain mutant proteins specifically suppress HB-EGF without cytotoxicity.

### Effects of Four R Domain Mutants on HB-EGF-Dependent Cell Growth in a Three-Dimensional (3D) Culture

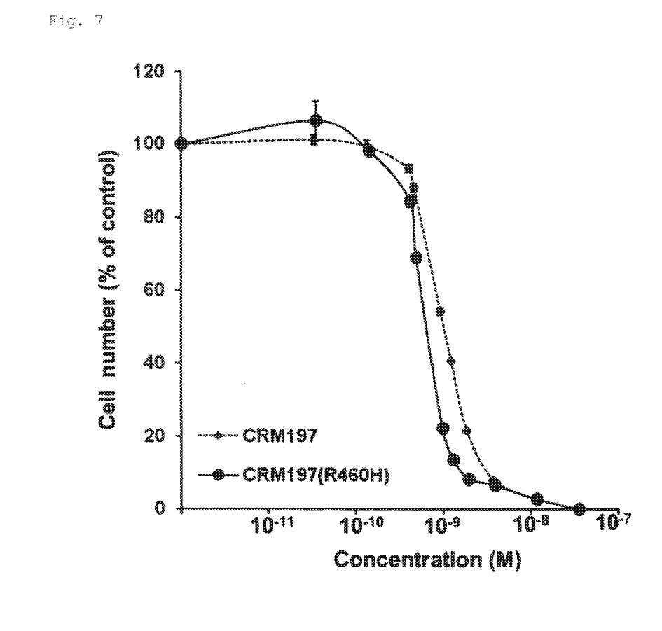

The effect of HB-EGF on cell growth is notably exerted particularly in 3D culture conditions (Mizushima, H., et al., J Cell Sci, 2009. 122 (Pt 23): p. 4277-86.). Thus, the present inventor cultured BRL cells in 3D culture conditions using a collagen gel to investigate the inhibitory activity of four R domain mutants against the HB-EGF-dependent growth of BRL cells. The four R domain mutants, i.e., MBP-R (H391K), MBP-R (G466D/D467A/V468T), MBP-R (R460H), and MBP-R (R472K), more effectively suppressed recombinant HB-EGF-induced BRL cell growth than MBP-R (WT) (FIGS. 6A to 6C, and Table 2). Further, compared with MBP-R (WT), these mutants more strongly suppressed the growth of BRLH cells that overexpress exogenous proHB-EGF (FIGS. 6D and 6E, and Table 4). These results show that the inhibitory activity of the four R domain mutants that was higher than that of MBP-R (WT) is not specific to a particular cell type or culture conditions.

### Affinity Constants of R Domain Mutants for HB-EGF

To investigate whether four R domain mutants possess increased affinity for HB-EGF compared with the wild-type R domain, the affinity constants of the mutants were determined using Biacore according to the manufacturer's instructions. The binding of each MBP-R domain protein to recombinant HB-EGF captured on the sensor tips was measured to calculate the affinity constants KA from the association rate constants and the dissociation rate constants. The affinity constant values of the proteins were as follows: CRM197, 9.22×10⁸ M⁻¹; MBP-R (R460H), 6.09×10⁸ M⁻¹; MBP-R (G466D/D467A/V468T), 4.44×10⁸M⁻¹; MBP-R (R472K), 4.42×10⁸ M⁻¹; MBP-R (H391K), 3.17×10⁸ M⁻¹; MBP-R (WT), 2.99×10⁸ M⁻¹ (Table 4). The affinity constant values almost correlate with the ID₅₀ values. These results show that the increased affinity for HB-EGF contributes to the inhibitory activity of the four mutants.

### Inhibitory Activity of CRM197 Mutant Against HB-EGF

MBP-R (WT) exhibits cell growth inhibitory activity lower than that of full length CRM197, suggesting that the overall molecular structure is required to show an absolute inhibitory effect. Thus, the present inventor reconstructed and prepared full length CRM197 having R460H mutation in the C. diphtheriae strain as described above and compared its inhibitory activity with that of CRM197. CRM197 (R460H) inhibited the HB-EGF-dependent growth of DER cells more effectively than the wild-type CRM197 (FIG. 7). The ID₅₀ values of CRM197 and CRM197 (R460H) were 1.02 nM and 0.72 nM, respectively (Table 5).

To investigate whether the affinity constant of CRM197 is improved by the R460H mutation as observed in the MBP-R domain mutants, the affinity constants KA of CPM197 and CPM197 (P4 60H) were compared using Biacore. The affinity constants KA of CRM197 and CRM197 (R460H) were 9.80×10⁸M⁻¹ and 1.63×10⁹M⁻¹, respectively (Table 5). These results show that the inhibitory activity of CRM197 against HB-EGF is increased by the mutation.

TABLE 1
Oligonucleotide sequences used for construction of
R domain mutants

| MBP-R Mutant | Forward Primer |
|---|---|
| MBP-WT | gcaggatcccccgcgtattctccgggg |
| | (SEQ ID NO: 7) |
| MBP-R(H391K) | caaccatttcttaaagacgggtatgctgtc |
| | (SEQ ID NO: 8) |
| MBP-R(G466D/ | cgttgcagagctatagacgatgcgaccactttttgt |
| D467A/V468T) | cgccctaaatc (SEQ ID NO: 9) |
| MBP-R(R460H) | cggaaaataaggatgcattgcagagctatagac |
| | (SEQ ID NO: 10) |
| MBP-R(R472K) | gtaactttttgtaaacctaaatctcctg |
| | (SEQ ID NO: 11) |
| MBP-R(D507N) | gaaatttcgtcgaactccataggcgttc |
| | (SEQ ID NO: 12) |
| MBP-R(H520K) | cagaaaacagtagataaaaccaaggttaattctaag |
| | (SEQ ID NO: 13) |

Complementary oligonucleotides of the Fw primers used were used as reverse primers.

TABLE 2
MBP-R mutants having a single amino acid substitution and their
inhibitory activity

| CRM197 or MBP-R Mutant | ID₅₀ [nM] |
|---|---|
| CRM197 | 1.2 |
| MBP-WT | 7.9 |
| MBP-R (H391K) | 3.7 |
| MBP-R (R460H) | 2.5 |
| MBP-R (V468T) | 6.2 |
| MBP-R (R472K) | 3.2 |
| MBP-R (D507N) | 4.5 |
| MBP-R (H520K) | 6.0 |

TABLE 3
MBP-R mutant having multiple amino acid substitutions and its
inhibitory activity

| MBP-R Mutant | ID₅₀ [nM] |
|---|---|
| CRM197 | 1.2 |
| MBP-R(WT) | 7.9 |
| MBP-R(G466D/D467A/V468T) | 2.6 |

TABLE 4
Comparison of MBP-R mutants and increased inhibitory activity

| | DER cell | | BRL | | BRLH | | | |
|---|---|---|---|---|---|---|---|---|
| | assay | | cell assay | | cell assay | | Biacore | |
| | ID₅₀ | Relative | Inhibition | Relative | Inhibition | Relative | KA | Relative |
| Name | (nM) | values | (%)* | values | (%)* | values | (M⁻¹) | values |
| CRM197 | 1.2 | | 57.8 | | 31.1 | | 9.22 × 10⁸ | |
| MBP-R (WT) | 7.9 | 1 | 77.7 | 1 | 86.9 | 1 | 2.99 × 10⁸ | 1 |
| MBP-R (H391K) | 3.1 | 2.5 | 61.3 | 1.27 | 81.7 | 1.06 | 3.17 × 10⁸ | 1.1 |
| MBP-R (G466D/ | 2.6 | 3 | 54.8 | 1.42 | 55.6 | 1.56 | 4.44 × 10⁸ | 1.5 |
| D467A/V468T) | | | | | | | | |
| MBP-R (R460H) | 2.5 | 3.2 | 60.4 | 1.29 | 47.4 | 1.83 | 6.09 × 10⁸ | 2 |
| MBP-R (R472K) | 3.2 | 2.5 | 62.1 | 1.25 | 47.5 | 1.83 | 4.42 × 10⁸ | 1.5 |

*Regarding HB-EGF inhibition, the number of cells in the case

where 5 μM of CRM197 or an MBP mutant was added was indicated as a percentage (%), based on the number of cells in the case where neither CRM197 nor an MBP mutant was added being 100%.

TABLE 5
Comparison of CRM197 with CRM197 (R460H)

| | DER cell assay | | Biacore | |
|---|---|---|---|---|
| | | Relative | | Relative |
| Name | ID₅₀ (nM) | values | KA (M⁻¹) | values |
| CRM197 | 1.02 | 1 | 9.80 × 10⁸ | 1 |
| CRM197 (R460H) | 0.72 | 1.42 | 1.63 × 10⁸ | 1.66 |

1. A polypeptide comprising an R domain of CRM197, the R domain comprising at least one amino acid substitution selected from the group consisting of H391K, R460H, V468T, R472K, D507N, and H520K.

2. The polypeptide according to claim 1, wherein the R domain of CRM197 comprises at least one amino acid substitution selected from the group consisting of H391K; R460H; G466D, D467A, V468 and R472K.

3. The polypeptide according to claim 1, wherein the polypeptide is a modified CRM197 comprising a C domain, a T domain, and the R domain of CRM197.

4. (canceled)

5. (canceled)

6. A pharmaceutical composition comprising an antitumor effective amount of the polypeptide according to claim 1 and a pharmaceutical carrier.

7. (canceled)

8. (canceled)

9. (canceled)

10. The polypeptide according to claim 2, wherein the polypeptide is a modified CRM197 comprising a C domain, a T domain, and the R domain of CRM197.

11. The pharmaceutical composition of claim 6, further comprising an antitumor agent selected from taxol, taxotere, docetaxel, irinotecan, 25 topotecan, doxorubicin, etoposide, CPT-11, camptostar, epothilone, tamoxifen, methoxtrexate, 5FU, temozolomide, cyclophosphamide, SCH66336, R115777, L778, 123, BMS214662, Iressa, Tarceva, antibodies to EGF receptor (EGFR), Gleevec, intron, ara-C, Adriamycin, Cytoxan, gemcitabine, uracil mustard, chlormethine, 30 ifosfamide, melphalan, chlorambucil, pipobroman, triethylenemelamine, triethylenethiophosphoramine, busulfan, carmustine, lomustine, streptozocin, dacarbazine, floxuridine, cytarabine, 6-mercaptopurine, 6-thioguanine, fludarabine phosphate, pentostatin, vinblastine, vincristine, vindesine, bleomycin, dactinomycin, daunorubicin, epirubicin, idarubicin, mithramycin, deoxycoformycin, and mitomycin-C.

12. A method of treating a cancer in a subject comprising administering an antitumor effective amount of a polypeptide according to claim 1 to a subject in need of such treatment.

13. The method of claim 12, wherein the cancer is one of ovarian cancer, breast cancer, prostate cancer, stomach cancer, lung cancer, colorectal cancer, and pancreatic cancer.
